(19) United States
(12) Patent Application Publication
(10) Pub. No.: US 2003/0225279 A1
(43) Pub. Date: Dec. 4, 2003

(54) Stavudine Polymorphic Form 1 process

(76) Inventors: Simon Crawford Bristow, West Yorkshire (GB); Philip Michael Cocks, Hudderson (GB); Ronald Harland, Yardley, PA (US); Rajesh B. Gandhi, Plainsboro, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/269,358

(22) Filed: Oct. 11, 2002

Related U.S. Application Data
(63) Continuation of application No. 10/101,037, filed on Mar. 19, 2002.
Application No. 10/101,037 is a continuation of application No. 09/934,863, filed on Aug. 22, 2001.
(60) Provisional application No. 60/230,261, filed on Sep. 6, 2000.
(60) Provisional application No. 60/231,766, filed on Sep. 12, 2000.

(51) Int. Cl. A61K 31/513; C 07D 4 5/04

(52) U.S. Cl. 544/310

(57) ABSTRACT

A process for the formation of Stavudine Polymorphic Form I from a mixture comprising Polymorphic Form I and at least one of Polymorphic Forms II and III is disclosed using the technique of Solution-Enhanced Dispersion by Supercritical Fluids (SEDS). A solution of the mixture in isopropyl alcohol/water is introduced into a particle formation vessel with a supercritical fluid under controlled temperature and pressure whereby the supercritical fluid substantially simultaneously disperses and extracts the solvent from the solution forming discrete particles of Stavudine Polymorphic Form I. A preferred supercritical fluid is carbon dioxide.

### CROSS-REFERENCE TO RELATED APPLICATIONS

This is a non-provisional application which claims the benefit of provisional applications, U.S. S. No. 60/230,261, filed Sep. 6, 2000, and U.S. S. No. 60/231,766, filed Sep. 12, 2000.

### FIELD OF THE INVENTION

This invention relates to an improved process for obtaining Polymorphic Form I of the antiretroviral compound Stavudine, useful in the treatment of retroviral infections, particularly HIV infections.

### BACKGROUND OF THE INVENTION

Stavudine, also known as d4T, is approved by the U.S. Food & Drug Administration for the therapeutic treatment of patients infected with retroviruses. Stavudine chemically is 2′, 3′-didehydro-3′-deoxythymidine. The compound, a nucleoside reverse transcriptase inhibitor, and its preparation are disclosed, for example, in U.S. Pat. No. 4,978,655, issued Dec. 18, 1990. It is known that Stavudine is effective in the treatment of infections caused by retroviruses such as murine leukemia virus and human immunodeficiency virus, i.e. HIV; HTLV III/LAV virus (the AIDS virus). Stavudine has enjoyed notable commercial success since its introduction.

It is known that Stavudine exists in three polymorphic forms that differ in solubility, designated as Forms I, II and III, respectively. Forms I and II are anhydrous polymorphs whereas Form III is hydrated and is pseudopolymorphic with Forms I and II. Of the three forms, Form I is stable and shows no transformations to other polymorphic forms, thus demonstrating its greater thermodynamic stability relative to the other Forms.

The phenomenon of polymorphism, the capacity of a substance to occur in different crystalline forms in the crystalline solid state, is well known, as are its ramifications on the process of drug development. The various characteristics and properties of the polymorphic forms of a substance, e.g. shape, color, density, dissolution properties and the like, will make one or more specific polymorphic forms desirable over the others for production and/or pharmaceutical compounding. As a result, a very early step in the process of product development of a new pharmaceutical agent is the determination of whether it exists in polymorphic forms and, if so, which of such forms possesses advantages for development of the eventual commercial pharmaceutical. In the instance of Stavudine, Form I has been found to be the most thermodynamically stable form, with no tendency for solid state conversion to the other Forms. Hence, it is Polymorphic Form I of Stavudine that is offered commercially under the trademark Zerit®.

U.S. Pat. No. 5,608,048, issued Mar. 4, 1997, teaches a process whereby Polymorphic Form I of Stavudine is prepared in substantially pure form from a mixture containing it in combination with one or more of Polymorphic Forms II and III. This process involves dissolving the mixture under anhydrous conditions in an organic solvent to form a saturated solution at a temperature of at least about 65° C. and continuously cooling the solution with stirring until precipitation of Stavudine Polymorphic Form I is completed. A requirement of the process, however, is that the rate of cooling cannot exceed about 20° C. per hour until the temperature falls below 40° C. In a preferred embodiment, the temperature is reduced about 10° C. over 15 minutes, then held for an hour and the steps repeated until the solution temperature falls below 40° C. There are disclosed further embodiments consisting of gradients in cooling the solution of the mixture of Polymorphic Forms.

The solvent utilized in the process described in U.S. Pat. No. 5,608,048 is selected from the group of methanol, ethanol, n-propanol, isopropanol, acetonitrile and ethyl acetate. It is emphasized, as stated previously, that the process must be carried out under anhydrous conditions. It will be appreciated that this process suffers from a number of disadvantages, among which are strict requirements in time and temperature management and control as well as strict moisture control. In accordance with the present invention, a method has been found whereby Stavudine Polymorphic Form I can readily be produced without such strict process control requirements.

### SUMMARY OF THE INVENTION

Stavudine Polymorphic Form I is produced in high yield and purity in a dry particulate form from a mixture comprising it and at least one of Polymorphic Forms II and III by a Solution-Enhanced Dispersion by Supercritical Fluids (SEDS) technique utilizing a particular solvent mixture as a vehicle. The process is carried out at constant temperature, without the requirement of constant stirring and further without the need for filtration and drying steps.

### DETAILED DESCRIPTION OF THE INVENTION

Solution-Enhanced Dispersion by Supercritical Fluids is a recognized technique known under the trademark SEDS, owned by Bradford Particle Design Limited, Bradford, West Yorkshire, England. It is described, for example, in U.S. Pat. No. 5,851,453, issued Dec. 22, 1998. The process is advantageous in that it can be utilized to control the polymorphic form of a drug substance in a single processing step. This control is achieved by operating the SEDS process under varied process parameters, primarily temperature, solvent composition and rate of crystallization until optimum conditions are determined for the desired polymorphic form. Particles produced utilizing the SEDS technique are free from static charge and contain only trace amounts of residual solvent. A further advantage of the process is that the particles are formed dry, thus eliminating the need for filtration and solvent removal, the latter being of particular advantage in terms of both cost and environmental considerations.

In the SEDS process, a solution of the material of interest is introduced into a chamber, designated a particle formation vessel, through a specially designed nozzle under stable conditions of temperature and pressure in combination with a supercritical fluid. The nozzle is essentially a coaxial design or the equivalent that produces a mixing of the two fluids being introduced at the point where they enter the chamber. The supercritical fluid mixes with, disperses and rapidly extracts the solvent from the solution. The insolubility of the solute in the supercritical fluid-solvent mixture induces the formation of particles by an antisolvent precipitation mechanism. By manipulating the various working parameters of pressure, temperature, solution concentration and flow rates in the nozzle, it is possible to control the size, shape and morphology of the product particles formed in the vessel. The co-introduction of the solution or dispersion of the desired substance and the supercritical fluid into the particle formation vessel creates, substantially simultaneously, dispersion and extraction of the vehicle or solvent by the action of the supercritical fluid. As the dissolved material is freed of solvent and dispersed by the supercritical fluid at the same time, the process produces discrete, dry particles that are retained in the vessel.

As utilized herein, the term “supercritical fluid” means a fluid substantially at or above its critical pressure (Pc) and critical temperature (Tc) simultaneously. As a practical matter in the application of the process in accordance with the present invention, the pressure of the fluid is likely to be in the range 1.01 Pc-7.0 Pc, preferably substantially above the Pc of the fluid, and the temperature in the range 1.01 Tc-4.0 Tc, preferably slightly above the Tc of the fluid. Suitable chemicals that can be utilized as supercritical fluids in the process of the present invention include carbon dioxide, nitrous oxide, sulfur hexafluoride, xenon, ethylene, chlorotrifluoromethane, ethane and trifluoromethane. In is essential to the efficient operation of the process that the supercritical fluid be an antisolvent for the desired product. Particularly preferred for the present process is supercritical carbon dioxide since Stavudine is practically insoluble therein.

In accordance with the process of the present invention, the supercritical fluid, preferably carbon dioxide, and a solution or dispersion of the material to be produced are introduced into the particle formation vessel through a coaxially designed nozzle as described in detail in U.S. Pat. No. 5,851,453. In the instance of the present process, the supercritical fluid is co-introduced with a solution comprising a mixture of Stavudine Polymorphic Form I and at least one of Polymorphic Forms II and III as formed in the synthesis thereof described in U.S. Pat. No. 5,608,048 utilizing thymidine as the starting material. The synthetic route for the preparation of Stavudine as described in said patent does not form part of the process of the present invention and will not be discussed in detail herein. Further, although the synthetic route disclosed in said patent is preferred, the particular pathway utilized to produce a mixture of Stavudine Polymorphic Form I and at least one of Forms II and III to be treated in accordance with the process of the present invention is not critical thereto. It must be borne in mid that, while the mixture of polymorphic forms of Stavudine to be treated in accordance with the present invention is in a purified state, it typically will contain entrained solvent, for example, DMSO, toluene and the like, as well as other impurities resulting from the synthesis. It has been found that such impurities, particularly the entrained solvents, are materially removed in accordance with the subject process. Even synthetic impurities, particularly those that are non-polar and will dissolve in the supercritical fluid, are likewise removed by the process of the present invention.

The solution containing a mixture comprising the polymorphic forms of Stavudine as described above and the supercritical fluid are co-introduced into the particle formation vessel such that there is instantaneous mixing of the two at the point of entry. The supercritical fluid is introduced under pressure and at a high flow rate in comparison to the solution containing the mixture of Stavudine polymorphic Forms. While not wishing to be bound by any particular theory or explanation of the phenomena taking place within the vessel, it is believed that the high velocity supercritical fluid causes the solvent of the solution to be broken up into droplets or other analogous fluid elements from which the vehicle/solvent is substantially simultaneously extracted by the supercritical fluid and dispersed, thereby resulting in the formation of discrete particles of the solid previously held in solution. Further, the high shearing action of the high velocity supercritical fluid ensures both dispersion of the vehicle/solvent and thorough mixing with the supercritical fluid thereby causing substantially immediate extraction thereof with the resultant formulation of discrete, dry particles of Stavudine Polymorphic Form I.

As described in U.S. Pat. No. 5,851,453, the nozzle utilized to introduce the supercritical fluid and the solution of Stavudine Polymorphic Forms into the vessel may be configured in various ways to achieve optimum mixing and dispersion. For example, an axial nozzle having three passages can be utilized to introduce a flow of the solution sandwiched between an inner and an outer flow of the supercritical fluid to achieve enhanced dispersion and, hence, greater control over, and uniformity of, the particle size of Stavudine Polymorphic Form I. Regardless of the configuration of the nozzle, at least one of the passages therein carries a flow of the solution and at least one of the passages carries a flow of the supercritical fluid.

The particle formation vessel is equipped with a retention means, such as a fine mesh screen, to catch and hold the particles of Stavudine Polymorphic Form I as they are formed therein. The apparatus is typically equipped at its outlet with a back-pressure regulator to maintain the particle formation vessel at the required operating pressure. The effluent from the back-pressure regulator is fed into a separator where it is decompressed to the gaseous state so that it may be recycled into the system if desired. The solvent for the solution will also separate as a liquid and may be collected and recycled, utilized in other applications or discarded. The system may be operated continuously or in a batch mode. When a sufficient amount of particles is collected in the vessel, the flow of solution is discontinued and the particles are dried by continued flushing with only pure supercritical fluid and then removed. A system may be operated with two particle formation vessels so that, while particles are being collected from one and it is being flushed and prepared to receive a renewed flow of solution, the other is producing. As those of ordinary skill in the art will appreciate, running the two vessels out of phase as described will assure continuous production.

The benefits of the process of forming Stavudine Polymorphic Form I in accordance with the process of the present invention are that it can be run isothermally, hence multiple depressurizing and pressurizing steps are not required, there is a considerable time saving in the eliminating of the drying and solvent removal steps and there is less likelihood of exposure of workers to reagents, particularly solvents, during the recovery step. In addition, the time-consuming and temperature control-sensitive technique previously utilized is no longer required. The same is true of the requirement in the previous process that the solution of the mixture of Polymorphic Forms of Stavudine be constantly stirred during the cooling process. The process of the present invention affords Stavudine Polymorphic Form I in higher yield than has heretofore been realized and in higher purity. The higher purity is possible since the present process removes a higher percentage of entrained solvents, including residual solvents from the synthesis. The particles of Stavudine Polymorphic Form I formed in accordance with the present process contain less than 100 ppm of entrained solvents. The particles size of Stavudine Polymorphic Form I formed in accordance with the present process is also advantageous over that previously available since the particles have an average size of from about 20 to about 40 microns whereas those from the previous manufacturing process range up to about 200 microns. Stavudine Polymorphic Form I formed by the present process is more polymorphically stable than that formed by the previous process as a result of the reduction in residual isopropyl alcohol and synthesis solvents since residual solvents have been shown to induce solid state transition upon storage.

In accordance with the process of the present invention, it has been found that a particular solvent combination for dissolution of a mixture of Stavudine Polymorphic Form I and at least one of Form II and Form III yields Form I in very high yield and purity in the SEDS process. Experiments were conducted with a wide variety of solvent combinations and process conditions and, as a result, it was determined that a mixture of isopropyl alcohol and water provided the optimum results. It has been found in accordance with the present invention that the presence of water in the solvent is essential since in the absence of water, all solvents tested as possible vehicles for the subject process produced Stavudine Polymorphic Form II to a significant degree.

The solution to be processed in accordance with the present invention preferably contains from about 0.1% to about 2%, most preferably about 1%, weight to volume of the mixture of Polymorphic Forms of Stavudine in a solvent mixture preferably from about 96:4 to 94:6, most preferably about 95:5, volume to volume isopropanol and water. The flow rates into the particle formation vessel are preferably a ratio of Stavudine solution to supercritical fluid of from about 0.005:1.0, most preferably about 0.02:1.0. The temperature and the pressure in the particle formation vessel are controlled during the process such that the temperature is above the Tc of the supercritical fluid and the pressure is substantially above the Pc of the supercritical fluid. Using carbon dioxide as the supercritical fluid, the temperature in the vessel is preferably from about 31.4 to 50° C., most preferably about 35° C., and the pressure is preferably from about 80 to 115 bar, most preferably about 90 bar.

In order to illustrate the unexpected nature of the solvent combination of the present invention in the SEDS process, starting with identical mixtures of the polymorphic forms of Stavudine, a solvent mixture of isopropyl alcohol and water containing 10% by volume water produced predominately Stavudine Polymorphic Form III; a solvent mixture containing 7.5% by volume water produced a mixture of Forms I and II; and a solvent mixture containing 2.5% by volume produced predominately Form II, all by analogous SEDS techniques. In view whereof, it is considered unexpected that the solvent combination of the invention produces pure Stavudine Polymorphic Form I. It is also considered unexpected that Form I can be produced by a process involving rapid cooling since the previous process is dependent on slow cooling under very controlled conditions with constant stirring. Further in view of the fact that U.S. Pat. No. 5,608,048 teaches that Stavudine Polymorphic Form I can only be produced under strict anhydrous conditions, it is considered unexpected that any combination of solvents containing water will even produce Form I. There is certainly no teaching in the patents discussed above that would suggest that, in the SEDS process, a solvent combination containing water would yield Stavudine Polymorphic Form I in high yield and high purity.

It is understood that various other embodiments and modifications in the practice of the invention will be apparent to, and can be readily made by, those of ordinary skill in the art without departing from the scope and spirit of the invention as described above. Accordingly, it is not intended that the scope of the claims appended hereto be limited to the exact description set forth above, but rather that the claims be construed as encompassing all of the features of patentable novelty that reside in the present invention, including all the features and embodiments that would be treated as equivalents thereof by those skilled in the relevant art. The invention is further described with reference to the following experimental work.

### Example 1

The following series of experiments was conducted in an SEDS apparatus such as described in U.S. Pat. No. 5, 851,453 and consisting essentially of a particle formation vessel, an axial configured double nozzle connected to a source of test solution under pressure and a source of carbon dioxide supercritical fluid under pressure, a means for particle retention within the vessel, a back-pressure regulator and a separator for separating the solvent from the carbon dioxide which reforms as a gas under reduced pressure. Various solvents were utilized to dissolve a mixture of Polymorphic Forms I, II and III of Stavudine. The results are shown in Table I.

TABLE I

| | | Sol. | | Solution | CO₂ | DSC | | | SEM |
|---|---|---|---|---|---|---|---|---|---|
| Press | Temp | Conc. | | Flow | Flow | Peak | XRPD | Yield | Morphology |
| bar | ° C. | w/v % | Solvent | mlmin⁻¹ | Mlmin⁻¹ | ° C. | Forms | % | Description |
| 200 | 35 | 5 | MeOH | n/a | 1 | — | II | 18 | Acicular, |
| | | | | | | | | | some >600μ |
| 200 | 35 | 3 | IPA | n/a | 1 | — | I & II | 63 | Acicular, |
| | | | | | | | | | some >600μ |
| 120 | 35 | 1 | Aceton | 0.3 | 20 | — | II | 62 | Acicular, |
| | | | | | | | | | up tp 100μ |
| 120 | 70 | 1 | Aceton | 0.3 | 18 | 171.5 | II | 47 | Acicular, |
| | | | | | | | | | 10-20μ |
| 250 | 35 | 1 | Aceto | 0.2 | 10 | 172.3 | II | 27 | Plates |
| | | | nitrile | | | | | | up tp 100μ |
| 80 | 70 | 1 | Aceto | 0.2 | 10 | 172.6 | I & II | 50 | Plates |
| | | | nitrile | | | | | | >100μ |
| 250 | 35 | 1 | IPA | 0.2 | 10 | 172.7 | II | 36 | Acicular/ |
| | | | | | | | | | Plates |
| | | | | | | | | | some >300μ |
| 80 | 70 | 10 | IPA | 0.2 | 10 | 167.4 | II | 33 | Plates >20μ |
| 150 | 50 | 8 | H₂O | 0.03 | 8 | | | 0 | No product |
| 80 | 90 | 2 | IPA | 0.3 | 15 | 168.6 | I & II | 68 | Plates, |
| | | | | | | | | | 20-30μ |
| 80 | 120 | 20 | IPA | 0.3 | 15 | — | I & II | 62 | Plates, |
| | | | | | | | | | 20-30μ |
| 85 | 120 | 1 | Aceto- | 0.3 | 15 | — | I & II | 41 | Plates, |
| | | | nitrile | | | | | | 20-30μ |
| 90 | 90 | 0.75 | Ethyl | 0.3 | 15 | — | II | 52 | Acicular, |
| | | | Acetate | | | | | | Part. 1-3μ |
| 80 | 90 | 1 | EtOH | 0.3 | 15 | — | II | 53 | Fused |
| | | | | | | | | | plates >10μ |
| 80 | 90 | 1 | IPA | 1 | 10 | — | — | 0 | No product |
| 250 | 35 | 1 | IPA | 1 | 10 | — | — | 0 | Trace |
| 250 | 40 | 1 | t-BuOH | 0.3 | 15 | — | — | 0 | Abandoned |
| 250 | 35 | 5 | DMSO | 0.3 | 15 | — | II | 33 | Plates, |
| | | | | | | | | | 20-30μ |
| 150 | 35 | 1 | EtOH | 0.4 | 9 | 170.8 | II | 68 | Large Rods |

In Table I, the DSC peak was obtained by accurately weighing a sample of between 2 and 5 mg and scanning it in a pierced, crimped aluminum pan by differential scanning calorimetry (DSC7, Perkin Elmer Ltd., UK). Since the melting points of the three polymorphic forms of Stavudine are very similar, this method was not utilized to determine the polymorphic form of the product. Polymorphic form was determined by X-ray powder diffraction (XRPD) using a Siemens model D-5000 diffractometer. Test samples were ground to a fine powder, using a mortar and pestle. The random orientation of the resulting crystallites ensures that every possible reflection place was represented parallel to the specimen surface. Data was collected between 1.5°, and 40° 2┘, using CuK₄ with a step increase of 0.05° and count intervals of three seconds. The SEM observation was carried out using a Hitachi S-520 electron microscope. Small quantities of sample were affixed to aluminum SEM stubs, coated with a conducting layer of gold and examined under a range of magnifications. The resulting micrographs were used to determine and describe particle morphology and estimate particle size.

The results shown in Table I demonstrate that, of the range of solvents and conditions tested, none was optimum for producing Stavudine Polymorphic Form I.

### Example 2

The following experimental runs were conducted utilizing mixtures of isopropanol (IPA) and water. All runs were conducted at a vessel temperature of 35° C., a flow of supercritical carbon dioxide of 9 mlmin⁻¹ and, with the exception of the final two runs that were conducted at a solution flow rate of 0.3 mlmin⁻¹, all runs were at a solution flow rate of 0.2 mlmin⁻¹. The results are reported in Table II.

TABLE II

| | Sol. | % H₂O | DSC | | | | |
|---|---|---|---|---|---|---|---|
| Press. | Conc. | plus | Peak | XRPD | Yield | SEM | |
| Bar | % w/v | IPA | ° C. | Form | % | Morphology | Comments |
| 120 | 1 | 10% | 168.5 | III | 37 | Acicular >500μ | Weight loss 2.94% |
| 120 | 1 | 10% | 168.7 | III | 43 | Mix of large % | Particles different than |
| | | | | | | small acicular | previous run |
| | | | | | | plates to 100μ | |
| 120 | 1 | 5% | 170.2 | I | 91 | Long, well- | Strong IPA smell in |
| | | | | | | defined needles | powder |
| | | | | | | 20-40μ | |
| 120 | 1 | 5% | 168.9 | I & II | 83 | Large acicular | Longer drying time |
| | | | | | | particles, some | than previous run. |
| | | | | | | chunks | Product was dry |
| 120 | 1 | 5% | 169.5 | I & II | — | Long, well- | Strong IPA smell, |
| | | | | | | defined needles | predominately Form I |
| 90 | 1 | 5% | 169.7 | I | 85 | Long, well- | Dry, similar |
| | | | | | | defined needles | morphology to starting |
| | | | | | | 20-100μ | material |
| 90 | 0.5 | 5% | 170.6 | I | 92 | Long, well- | Reduced solution |
| | | | | | | defined needles | conc. Similar to |
| | | | | | | 20-80μ | starting material |
| 90 | 0.5 | 5% | 170.6 | I | 87 | Long, well- | Reduced solution |
| | | | | | | defined needles | conc. Similar to |
| | | | | | | up to 100μ | starting material |
| 90 | 0.5 | 5% | 169.7 | I & II | 79 | Long, well- | Raised filter and |
| | | | | | | defined needles | utilized different |
| | | | | | | up to 150μ | nozzle |
| 90 | 1 | 5% | 170.2 | I | 95 | Long, well- | Original nozzle, no |
| | | | | | | defined needles | raised filter, increased |
| | | | | | | up to 100μ | conc.* |
| 90 | 0.5 | 5% | 169.6 | I | 75 | Long, well- | Original nozzle, no |
| | | | | | | defined needles | raised filter |
| | | | | | | up to 150μ | |
| 90 | 1 | 5% | — | I | 90 | Long, well- | 500 ml vessel used, no |
| | | | | | | defined needles | sign of Form II |
| | | | | | | up to 100μ | |
| 90 | 0.5 | 2.5% | — | II | 78 | Long | Water content reduced |
| | | | | | | needles/flat | for effect |
| | | | | | | bars, some >200μ | |
| 90 | 0.5 | 7.5% | — | I & II | 69 | Very long sharp | Water content |
| | | | | | | needles, some | increased for effect |
| | | | | | | >300μ | |
| 90 | 1 | 5% | — | I | 93 | | Increased throughput |
| | | | | | | | in 50 ml vessel |
| 90 | 1 | 5% | — | I | 90 | | 500 ml vessel used |

The run with the increased concentration (*) was conducted with a supercritical carbon dioxide flow rate of 10 mlmin⁻¹. The weight loss in the first run was determined using Thermogravimetric Analysis (TGA 7, Perkin Elmer Ltd.) by heating samples in open pans at 10° C. min⁻¹ between 25 and 200° C. These results demonstrate preferred conditions for producing Stavudine Polymorphic Form I in accordance with the subject process utilizing mixtures of 95:5 IPA and water.

### Example 3

Residual solvent analysis was performed on samples of Stavudine Polymorphic Form I prepared in accordance with the process of the present invention and commercial material that had not been processed in accordance with the present process. The analysis was performed using headspace-gas chromatography having the capacity to quantify residual isopropyl alcohol levels up to 2023 ppm using external standardization. Deionized water was utilized as the solvent as it is not detected by flame ionization detection, hence does not interfere with the analysis. Standard solutions of Stavudine Polymorphic Form I were prepared with concentrations up to 500 μgml⁻¹. Test samples solutions containing high Stavudine concentrations between 5 and 25 mg ml⁻¹ were prepared and tested in sealed vials in a Varian Star 3400cx with Flame Ionization Detector, Varian, UK. The results of analysis of the headspace in each sealed vial are given in Table III below.

TABLE III

| | | | | Total ppm |
|---|---|---|---|---|
| Processed | Total Sample | (μg) Equivalent | Actual Total | IPA in the |
| (Y/N) | Wt. in Grams | to 1 ppm | (μg) Present | Sample |
| Unprocessed | 0.04302 | 0.04302 | 10.326 | 240 |
| Unprocessed | 0.4828 | 0.04828 | 10.806 | 224 |
| Processed | 0.02013 | 0.02013 | 0.992 | 49 |
| Processed | 0.02 | 0.02 | 1.082 | 54 |
| Processed | 0.02419 | 0.02419 | 2.016 | 83 |

The benefit of the subject process in terms of residual solvent content of processed Stavudine Polymorphic Form I are clearly demonstrated by the data in Table III.

We claim:

1. A process for producing purified Stavudine Polymorphic Form I from a mixture comprising Stavudine Polymorphic Form I and at least one of Stavudine Polymorphic Forms II and III comprising:
a) forming a solution of said mixture in a solvent consisting of isopropyl alcohol and water in a volume ratio of from about 96:4 to 94:6;
b) simultaneously introducing said solution and a supercritical fluid into a particle formation vessel through a nozzle having coaxial passages that terminate at the point of entry into said vessel, at least one of said passages carrying a flow of said solution and at least one of said passages carrying a flow of said supercritical fluid, thereby causing said supercritical fluid to substantially simultaneously disperse and extract said solvent from the solution thus forming discrete particles of Stavudine Polymorphic Form I, said vessel being maintained at a temperature above the critical temperature of said supercritical fluid and a pressure substantially above the critical pressure of said supercritical fluid; and
c) recovering said particles from the vessel.

2. A process in accordance with claim 1, wherein the solvent is isopropyl alcohol and water in a volume to volume ratio of 95:5.

3. A process in accordance with claim 1, wherein said supercritical fluid is carbon dioxide.

4. A process in accordance with claim 1, wherein the solution contains from about 0.1 to about 2 percent, weight to volume of said mixture.

5. A process in accordance with claim 4, wherein said solution contains about 1 percent weight to volume of said mixture.

6. A process in accordance with claim 1, wherein the temperature in said particle formation vessel is from about 31.4 to 50° C.

7. A process in accordance with claim 6, wherein the temperature in the particle formation vessel is 35° C.

8. A process in accordance with claim 1, wherein the pressure in the particle formation vessel is between about 80 and 115 bar.

9. A process in accordance with claim 8, wherein the pressure in the particle formation vessel is about 90 bar.

10. A process in accordance with claim 1, wherein the flow ratio of said solution to said supercritical fluid into said particle formation vessel is from about 0.005:1.0 to 0.4:1.0.

11. A process in accordance with claim 10, wherein is the flow ratio of said solution to said supercritical fluid into said particle formation vessel is 0.02:1.0.

12. A process in accordance with claim 1 additionally including the steps of recovering and optionally recycling at least one of the solvent and the supercritical fluid following particle formation.

13. A process in accordance with claim 1, wherein said discrete particles are collected by discontinuing the flow of said solution into the vessel and removing the particles by flushing the vessel with supercritical fluid.

14. A process in accordance with claim 13, where two of said vessels are operated out of phase such that one is producing particles while particles are being collected from the other, thereby producing particles in a continuous manner.

15. Stavudine Polymorphic Form I characterized by having an average particles size of from about 20 to about 40 microns and enhanced Polymorphic stability as a result of having less than 100 ppm entrained solvents, formed in accordance with the process of claim 1.
